(19) United States
(12) Patent Application Publication
(10) Pub. No.: US 2026/0194567 A1
(43) Pub. Date: Jul. 9, 2026

(54) Systems and Methods for Improving Transformer Health

(71) Applicant: Saudi Arabian Oil Company, Dhahran (SA)

(72) Inventors: Abdallah Guraishi, Dhahran (SA); Rafat Rob, Dhahran (SA); Mansoor Zahrani, Dammam (SA)

(21) Appl. No.: 19/010,857

(22) Filed: Jan. 6, 2025

(51) Int. Cl. G01R 31/12; G01N 33/28

(52) U.S. Cl. CPC G01R 31/1263; G01N 33/2841

(57) ABSTRACT

Disclosed are methods, systems, and computer-readable medium to perform operations for real-time monitoring of the health of a transformer, where the operations involves obtaining real-time concentration measurements of a plurality of furanic compounds within the transformer; calculating, based on a first real-time concentration measurement of a first furanic compound and using more than one calculation approach, a degree of polymerization associated with the transformer; detecting, based on the degree of polymerization and the types of plurality of furanic compounds, an abnormal condition of the transformer; and responsively performing a remedial action to address the abnormal condition.

### TECHNICAL FIELD

The present disclosure relates to systems and methods for improving transformer health.

### BACKGROUND

Transformers are used in industrial systems to distribute electrical power efficiently, thereby ensuring that machinery and equipment operate at the correct voltage levels while minimizing energy loss over long distances. Like many electrical devices, however, transformers age with time. Transformer aging is primarily caused by the degradation of its insulation system, particularly the cellulose paper and insulating oil. Over time, factors like heat, moisture, and oxygen break down the cellulose, leading to reduced mechanical strength and dielectric performance. As the insulation deteriorates, the transformer becomes more prone to failures and reduced efficiency. Proper monitoring and timely maintenance are essential to extend transformer life and prevent costly outages.

### SUMMARY

One aspect of the subject matter described in this specification may be embodied in a method for real-time monitoring of the health of a transformer, where the method involves obtaining real-time concentration measurements of a plurality of furanic compounds within the transformer; calculating, based on a first real-time concentration measurement of a first furanic compound and using more than one calculation approach, a degree of polymerization associated with the transformer; detecting, based on the degree of polymerization and the types of plurality of furanic compounds, an abnormal condition of the transformer; and responsively performing a remedial action to address the abnormal condition.

The previously described implementation is implementable using a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium. These and other embodiments may each optionally include one or more of the following features.

In some implementations, the real-time concentration measurements are obtained from at least one local furanic sensor installed within the transformer.

In some implementations, the method is performed by an embedded system including a microcontroller, a local display, and a communication device.

In some implementations, the plurality of furanic compounds comprise: 2 Furaldehyde (2FAL), 5-Methyl-2-Furaldehyde (5M2F), 2-Acetylfuran (2ACF), 5 Hydroxymethyl-2-Furaldehyde (5H2F), 2-Furfuryl Alcohol (2FOL).

In some implementations, the first furanic compound is 2 Furaldehyde (2FAL).

In some implementations, the more than one calculation approach includes more than one of: a Chendong approach, a Stebbin approach, a first Myers approach, or a second Myers approach.

In some implementations, the Chendong approach includes calculating the degree of polymerization as:

$$D P = \frac{1.51 - {log}_{10} ( {C}_{fur} )}{0.0035} ,$$

where C_{fur} is the first concentration of 2FAL in parts per million (ppm).

In some implementations, the Stebbin approach includes calculating the degree of polymerization as:

$$DP = \frac{1.5 \; 655 - {log}_{10} ( {C}_{fur} )}{0.0035} ,$$

where C_{fur} is the first concentration of 2FAL in parts per million (ppm).

In some implementations, the first Myers approach includes calculating the degree of polymerization as:

$$DP = - 34 \; 3.8 * {log}_{10} ( {C}_{fur} ) + 1387.5 ,$$

where C_{fur} is the first concentration of 2FAL in parts per billion (ppb).

In some implementations, the second Myers approach includes calculating the degree of polymerization as:

$$DP = - 28 5.7 * {log}_{10} ( {C}_{fur} * 0.88 ) + 1288.6 ,$$

where C_{fur} is the first concentration of 2FAL in parts per billion (ppb).

In some implementations, the remedial action includes at least one of: removing the transformer from service, outputting an audible alert, outputting an alert on a display device, or adjusting operation of the transformer.

The details of one or more implementations of these systems and methods are set forth in the accompanying drawings and the description below. Other features, objects, and advantages of these systems and methods will be apparent from the description and drawings, and from the claims.

### DESCRIPTION OF DRAWINGS

FIG. 1 is an example real-time transformer health monitoring system.

FIG. 2 is a flow diagram of example microcontroller logic within the example real-time transformer health monitoring system.

FIG. 3 is a flow diagram for real-time transformer health monitoring.

FIG. 4 is a flow diagram of an example method.

FIG. 5 is a block diagram of an example computer system.

Like reference numbers and designations in the various drawings indicate like elements.

### DETAILED DESCRIPTION

This disclosure describes systems and methods for monitoring the health and age of a transformer in real-time. Transformer insulation issues, including the deterioration of both paper and transformer oil, account for a significant amount of transformer failures. Existing methods of inspecting the health and age of transformers require: (1) taking a transformer offline, and (2) manually extracting samples from the offline transformer's insulating paper to conduct laboratory tests on the extracted samples. This process is susceptible to variability and contamination that can lead to inaccurate lab results. This process also requires a complete shutdown of a transformer prior to any extraction of transformer oil samples. Additionally, periodic transformer shutdown and sample extraction produces inaccurate lab results since the rate of degradation and measured variables do not consistently change over time. Indeed, such conditions exhibit nonlinear variations. Factors such as working environment, temperature, impurities present in a transformer oil, and load factor contribute differently to insulation aging.

The disclosed real-time measuring and monitoring systems and methods for approximating the health and age of a transformer reduce errors in sample gathering and lab analysis. The disclosed systems and methods eliminate the need for a complete shutdown of a transformer before conducting tests regarding its health. Furthermore, the disclosed systems and methods provide real-time remedial actions to resolve identified errors or faults in the operation of a transformer. The real-time measurement and monitoring of a transformer's health allows for timely maintenance and reduces the likelihood of overdue maintenance, thereby lowering maintenance expenses, enhancing transformer longevity, improving operational efficiency and safety, and mitigating failure incidents. Compared to existing techniques, the disclosed calculations for evaluating the health of a transformer yield more accurate and consistent results by using the real-time monitoring system that provides daily measurements in real-time.

As described in more detail below, the disclosed systems and methods monitor a transformer's health (e.g., age) by detecting the presence of furanic compounds (Cfur) in the transformer oil. As the transformer lifespan is consumed, the transformer's insulating paper experiences degradation that releases furanic compounds into the transformer oil. Thus, there is a correlation between the concentration of furans and the age of a transformer. The furanic compounds are detected by sensors that provide real-time measurements of the concentration of furanic compounds. Due to the molecular motion of the transformer oil, the furanic compounds are uniformly dispersed in the oil as their movement is governed by Fick's Law. This even distribution of the furanic compounds in the transformer oil enables the disclosed systems and methods to automatically, and in real-time, provide online measurements from any point since the concentration of furanic compounds remains constant irrespective of location within the oil. In some examples, the concentration of the furanic compounds is used in more than one calculation approach to produce a degree of polymerization (DP) approximation for the transformer. The degree of polymerization is an important metric for the longevity and condition of insulation. Specifically, a higher DP value is indicative of a larger loss of remaining life within a transformer.

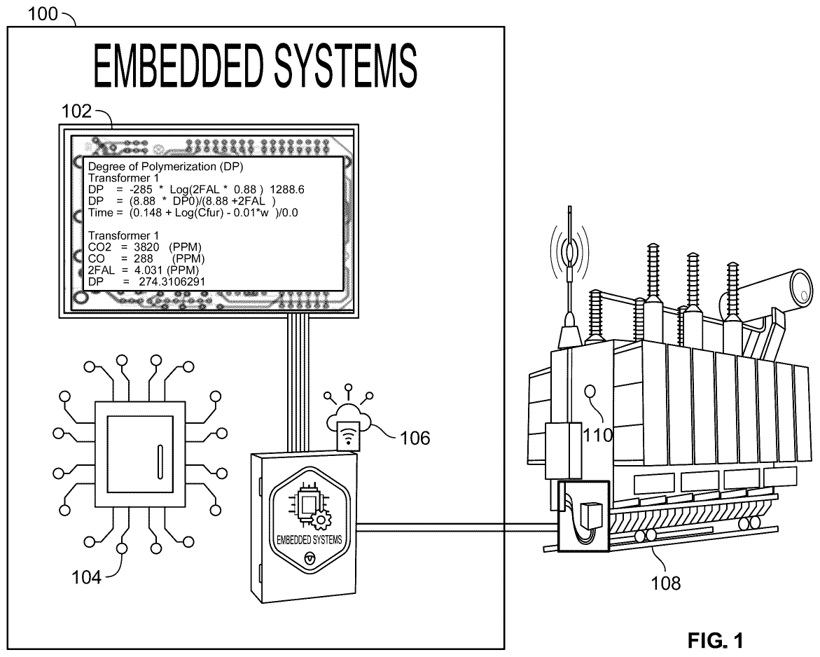

FIG. 1 is an example real-time transformer health monitoring system 100. As shown in FIG. 1, the real-time transformer health monitoring system 100 includes furanic sensors 110 for detecting furanic compound concentrations in a transformer 108. The furanic sensors 110 can be arranged on, within, or partially within the transformer 108. For purposes of FIG. 1, furanic sensors 110 are shown on transformer 108, but can also include a portion within the transformer 108. In this disclosure, the furanic sensors 110 are also referred to as Degree of Polymerization (DP) sensors. As also shown in FIG. 1, the real-time transformer health monitoring system 100 also includes a communication interface 106, a microcontroller 104, and a local display 102. Among other things, the communication interface 106 communicates with the furanic sensors 110 (e.g., to obtain furanic compound measurements), the microcontroller 104 handles the system 100's computer commands and computations, and the local display 102 displays a calculated DP.

In some implementations, the real-time transformer health monitoring system 100 also includes a solar power system (e.g., one or more solar panels and a battery) that power the system (e.g., the microcontroller 104 and/or the furanic sensors 110). In some examples, the real-time transformer health monitoring system 100 (or perhaps a subset of its components) are configured to enter a power-saving mode when measurements are not needed. The real-time transformer health monitoring system 100 can be configured to do so for energy conservation purposes. The microcontroller 104 can be further configured with a high-speed USB, wired communication interfaces (e.g., Ethernet), and/or wireless communication interfaces (e.g., cellular communication interfaces) to reduce human intervention and minimize errors during oil sampling, testing, and calculating the DP. Furthermore, the communication interface 106 can include wired and/or wireless communication interfaces for communicating with the furanic sensors 110 and/or other computing devices.

In some implementations, the microcontroller 104 is configured to execute instructions that initialize the real-time transformer health monitoring system 100. Additionally, the microcontroller 104 is configured to execute instructions that cause the real-time transformer health monitoring system 100 to monitor the health of the transformer 108.

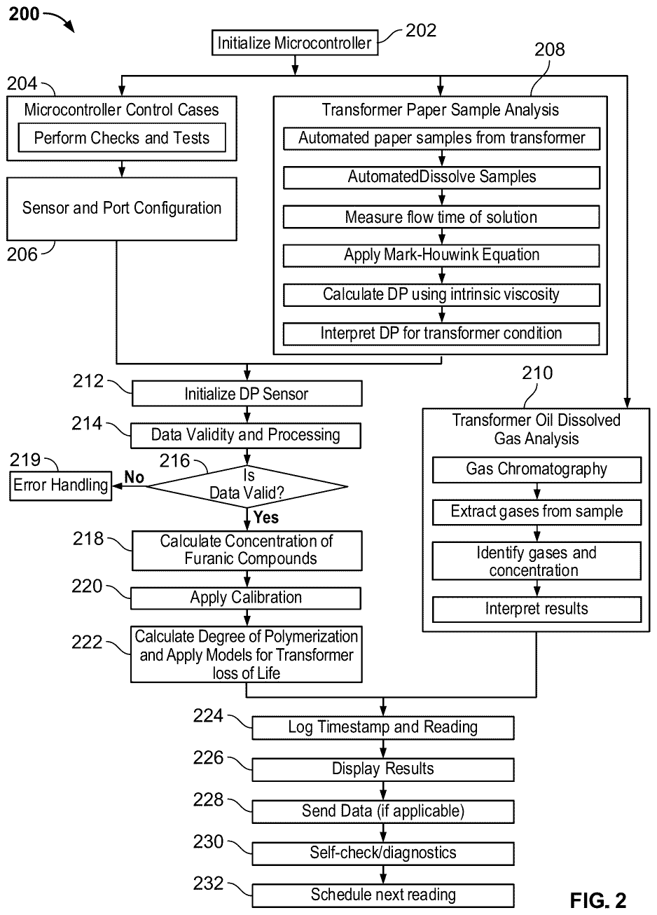

FIG. 2 is a flow diagram of example microcontroller logic 200 of the microcontroller 104. As shown in FIG. 2, the example microcontroller logic 200 starts at step 202. Step 202 involves initializing the microcontroller 104. Step 204 involves testing microcontroller control cases. Specifically, step 204 involves performing checks and tests. In some examples, the checks and tests include: (i) self-checking and diagnostics, (ii) verifying memory integrity, (iii) checking flash memory, (iv) checking random-access memory (RAM), (v) testing communication ports, and (vi) testing the sensor interface of the microcontroller 104. Step 206 involves sensor and port configuration. In this step, the clock system and the I/O ports are configured. Additionally, this step involves sensor initialization and checking fuses, power integrity, and sensor connections.

Step 208 involves transformer paper sample analysis. In some examples, this step is performed every time the microcontroller 104 is initialized-which could occur every measurement cycle, upon restarting the microcontroller 104, or when an initialization request to restart the process is received from a remote source, like a control room. In this step, paper samples from the transformer 108 are acquired automatically, perhaps using an automated mechanical paper sample extraction method. The instrument is capable of operating in the high voltage and high temperature conditions of the transformer 108, e.g., in the transformer oil where the sensors may be partially submerged.

In one example, step 208 involves executing an “AutomatedDissolve” function. Here, the extracted insulation paper sample is first transported through a sealed or insulated transfer line to an outside vacuum or pressurized analysis chamber to prevent oxidation or exposure to external contaminants. The sample is then dissolved by automatically injecting a solvent of appropriate volume, concentration, temperature, and exposure time to enable analysis of the sample. Once the insulation paper sample is fully dissolved, its viscosity is measured through an automated viscometer. This measurement reflects the change that may have taken place in the cellulose molecular structure. Reduction in viscosity corresponds to a reduced molecular weight and DP of the paper insulation.

In some implementations, step 208 further involves determining the molecular weight of the sample by using the viscosity in the Mark-Houwink equation. The Mark-Houwink equation provides the molecular weight of cellulose, which is indicative of the structural integrity of the insulating paper, in relation to its viscosity. The Mark-Houwink equation is:

$$\{ η \} = K {M}^{a} ,$$

where η=is the intrinsic viscosity of the dissolved cellulose sample, M=is the molecular weight of cellulose, and K and a are constants that depend on the solvent and temperature used. In the context of transformer insulation, molecular weight, M, is associated with the DP of the insulation paper's cellulose. As the insulation degrades, the DP drops as the chains of cellulose break and mechanical strength decreases. Values of K and a for known selected solvent/temperature conditions and intrinsic viscosity {η}can give the value of M which gives a quantitative assessment of insulation degradation. Then, the molecular weight obtained using the Mark-Houwink equation can be further related to the Degree of Polymerization.

Step 208 may also involve cleaning of the sensors and devices to prevent the accumulation of particles or residue that may adversely impact the accuracy of analysis. The cleaning may be manual by means of a button that forcibly removes fouling within the sensor, or it may be done by an automatic mechanism by introducing cleaning oil for removing any fouling.

In some implementations, step 210 involves performing a transformer oil dissolved gas analysis. Specifically, the transformer oil dissolved gas analysis involves continuously or periodically performing automated gas chromatography by an instrument configured to release dissolved gases from an oil matrix using negative pressure, vacuum, or inert sparging with gases (e.g., nitrogen or argon gases) without compromising sample integrity. Specifically, gases are extracted from the oil sample and then are transferred to a gas chromatograph with special columns optimized for hydrocarbon gases and COx compounds. High-performance column material systems such as packed or capillary columns may be used to enable the resolution of hydrocarbon gas species detection in the range of parts-per-million (ppm) sensitivity.

Then, the microcontroller 104 identifies gases present within the oil by using detectors that are configured as a combination of flame ionization detection (FID) and thermal conductivity detector (TCD) to enable dual-mode detection. FID is utilized for its hydrocarbon detection (e.g., CH₄ and C₂H₄) and TCD is utilized for its detection of H₂ and CO₂, thereby achieving comprehensive gas characterization. Identification of gases may also rely on advanced spectral analysis software that uses pattern recognition algorithms that identify each gas by retention time and detector response signature. By cross-referencing with embedded spectral libraries to confirm the identities of gases, ambiguities are eliminated in complex or overlapping chromatographic peaks.

In some implementations, step 208 also involves quantifying the gas concentration within the oil. The quantitative analysis allows for an extended dynamic range between low parts per billion (ppb) to high parts per million (ppm). This is enabled by a calibration curve for each gas, which considers the response of detectors to ensure accurate quantification over many possible fault conditions. Further, compensatory algorithms allow for real-time data correction algorithms, compensating for conditions such as pressure and ambient temperature fluctuation. This also ensures that the readings of concentration values reflect accurate quantity of dissolved gases within the transformer environment. Real-time gas-concentration data is compared on a continuous basis to historical baselines, allowing threshold-based notifications when the concentrations exceed levels set for fault conditions like overheating or dielectric degradation. Then, the result for transformer health is interpreted based on the concentration of gases detected, e.g., by identifying the corresponding effect that each gas is indicative of on the transformer 108 health.

In some implementations, step 210 also involves a calibration function in which the automatic system is pre-calibrated with accurate retention times for each gas. This calibration is usually temperature-controlled and may rely on internal or international standards to ensure very high accuracy in peak identification and ensure accurate identification of gases regardless of the complexity of gas mixtures. The interpretation of the result is performed by using a combination of models to analyze ratios of different gases and provide likely fault classification, including high temperature overheating due to high values of CO₂ and CO, partial discharge due to high H₂ and CH₄, or arcing due to the presence of C₂H₂. The interpretation of the gas chromatography step may be supplemented with a machine learning fault detection algorithm that relies on historical dissolved gas analysis data for the identification of intricate fault symptom patterns and adaptive thresholding.

Step 212 involves initialization of the DP sensor 110. In some examples, initialization of the DP sensor 110 includes: (i) configuring the sensor, (ii) reading sensor data, (iii) starting data acquisition, and (iv) reading raw data from sensor. Step 214 involves data validity and processing. In some examples, steps for data validity include: (i) calculating checksum, (ii) verifying checksum, and (iii) verifying validity of data. Step 219 is performed if either checksum or data is found to be invalid. In this step, an error is generated based on the type of error detected. Example errors that may result in performing step 219 include (i) sensor errors, (ii) data conversion errors, or (iii) communication errors.

Step 218 is performed if no errors are detected. This step involves calculating the concentration of the furanic compounds. The target furanic compounds include 2-Furaldehyde (2FAL), 5-Methyl-2-Furaldehyde (5M2F), 5-Hydroxomythyl-2-Furaldehyde (5H2F), 2-Acetyl Furan (2ACF), and 2-Furfuryl Alcohol (2FOL). The target furanic compounds correspond to relative parameters of a transformer's condition. Specifically, 2FAL corresponds to overheating and normal aging, 5M2F corresponds to high temperatures, 2ACF corresponds to rare and undefined causes, 5H2F corresponds to oxidation, and 2FOL corresponds to high moisture.

Step 220 involves applying calibration. Step 222 involves calculating the degree of polymerization and applying models for transformer loss of life. In some examples, the models for transformer loss of life include (i) Arrhenius Model, (ii) Pablo Model, (iii) Chendong Model, (iv) Stebbin Model, (v) a first Myers Model, (vi) a second Myers Model, (vii) Mark-Houwink Model, or (viii) a combination of the above models.

Step 224 involves logging the timestamps and readings. These logged datapoints may be used at later steps, such as step 228 below. Step 226 involves displaying the results. In particular, the results are displayed on the local display 102, and may additionally or alternatively be transmitted to a remote-control room that includes a central computing device. The displayed results may include the calculated DP, the measured furanic compound concentration, the concentration of CO₂ and CO that are derived from the transformer oil dissolved gas analysis, and the timestamps for the measured and calculated results.

Step 228 involves optionally sending the data to a remote server or a computer via wired or wireless communication. Step 230 involves performing self-checks and diagnostics. Step 232 involves scheduling the next reading. The system may engage in power-saving mode between scheduled measurements to conserve the overall energy consumption of the system.

Note that step 219 of error handling is performed if an error is detected at step 216. Step 219 can involve running diagnostic tests, restarting the system, or generating an alert in response to detecting an error.

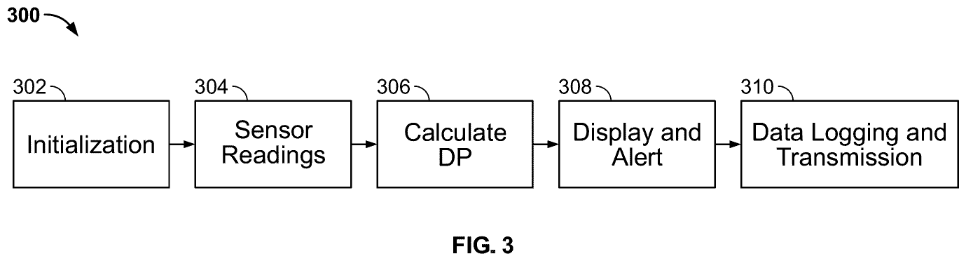

FIG. 3 is a flow diagram 300 for real-time transformer health monitoring. The process begins with the initialization step 302 wherein the microcontroller 104 performs initialization such as furanic sensor 110 configuration, microcontroller 104 port configuration, performing internal microcontroller checks and tests, and performing calibration.

The sensor reading step 304 uses the furanic sensors 110 to measure in real time the concentration of furanic concentration present in the transformer 108 oil. The furanic sensors 110 are configured to detect the presence of at least the following compounds: Furaldehyde (2FAL), 5-Methyl-2-Furaldehyde (5M2F), 2-Acetylfuran (2ACF), 5 Hydroxymethyl-2-Furaldehyde (5H2F), and/or 2-Furfuryl Alcohol (2FOL). These furanic compounds, e.g., at specified thresholds, can be indicative of a transformer's condition. Specifically, the presence of 2FAL can be indicative of overheating and normal aging, 5M2F can be indicative of high transformer temperatures, 2ACF can be indicative of rare and undefined causes, 5H2F can be indicative of oxidation, and 2FOL can be indicative of high moisture. After the furanic sensors 110 measure the concentration of the furanic compounds in the transformer 108 oil, the furanic sensors 110 transmit the measurements to the communication interface 106. These received measurements are read and used as an input by the microcontroller 104 in the following step.

In some implementations, the sensor reading step 304 additionally involves performing a dissolved gas analysis within the transformer 108 oil to detect the presence of gases such as carbon dioxide (CO₂) and carbon monoxide (CO).

In some implementations, step 306 of calculating DP uses the measurements from the furanic sensor 110 in a plurality of DP calculation approaches. These approaches include a Chendong approach, a Stebbin approach, a first Myers approach, a second Myers approach, an Arrhenius approach, and/or a Pablo approach.

In some examples, the Chendong approach may be used to calculate the DP of the transformer 108. The Chendong approach is expressed as

$$DP = \frac{1.51 - {log}_{10} ( {C}_{fur} )}{0.0035} ,$$

where C_{fur} is the first concentration of 2FAL in parts per million (ppm).

Additionally and/or alternatively, the Stebbin approach may be used to calculate the DP of the transformer 108. The Stebbin approach is expressed as

$$D P = \frac{1.5655 - {log}_{10} ( {C}_{f u r} )}{0.0035} ,$$

where C_{fur} is the first concentration of 2FAL in parts per million (ppm)

Additionally and/or alternatively, the first Myer approach may be used to calculate the DP of the transformer 108. The first Myer approach is expressed as

$$D P = - 343.8 * {log}_{10} ( {C}_{f u r} ) + 1387.5 ,$$

where C_{fur} is the first concentration of 2FAL in parts per billion (ppb).

Additionally and/or alternatively, the second Myer approach may be used to calculate the DP of the transformer 108. The second Myer approach is expressed as

$$D P = - 285.7 * {log}_{10} ( 2 FAL * 0.88 ) + 1288.6 ,$$

where 2FAL is measured in parts per billion (ppb).

Additionally and/or alternatively, the Arrhenius model approach may be used to calculate the DP of the transformer 108. The Arrhenius model approach is expressed as

$$K = A \cdot {e}^{\frac{{E}_{a}}{RT}}$$

where K is the rate constant, A is a pre-exponential factor (or the frequency of collisions which lead to reaction), Eₐ is the activation energy needed for reaction, R is the gas constant, and T is the absolute temperature.

Additionally and/or alternatively, the Mark-Houwink model approach may be used to calculate the DP of the transformer 108. The Mark-Houwink model approach is expressed as

$$\{ η \} = {KM}^{a}$$

where η is the intrinsic viscosity of the dissolved cellulose sample, M is the weight of cellulose, and K and a are both constants that vary based on the solvent and temperature used.

In the display and alert step 308, the microcontroller 104 displays the results of the above approaches for calculating the DP of the transformer on a display 102 that is installed locally near the respective transformer. The microprocessor 104 may also display additional values such as the measured furanic compound concentration, the concentration of the CO₂ concentration within the transformer 108 oil, the concentration of CO concentration within the transformer 108 oil, the concentration, or a combination of the above.

In some implementations, the microcontroller 104 issues alerts and displays a diagnostic alert message on the local display 102 upon detecting certain fault conditions. These conditions include determining that the transformer 108 health exceeds normal conditions, the data measured by the furanic sensors 110 is found to be invalid, a communication error is detected, or if a fault prevents the real-time measurements of the furanic compounds present in the transformer 108 oil. The microcontroller 104 may monitor faults by applying statistical methods, including signal smoothing and/or noise reduction algorithms to filter out all invalid data points and limit data to valid and reliable information which contribute to the computation of DP. The microcontroller 104 may also monitor faults by comparing predefined operational threshold values with key indicators such as concentration of furanic compounds, moisture content, and temperature fluctuations.

In the event of a severe degradation of transformer insulation where the DP value drops below a critical pre-set threshold, an emergency procedure may be performed. This emergency procedure may consist of delivering an alarm signal to an operator, actuating external controls, and conducting an automatic controlled shutdown of the transformer 108 to prevent a catastrophic failure and further degradation of insulation.

In some cases, the output of the microcontroller's 104 DP calculation and fault alerts may be transmitted to a remote location such as a remote control room to provide immediate monitoring of the transformer 108 condition in in addition to the local monitoring of each transformer's 108 local display 102.

In the data logging and transmission step 310, the measured DP value for the transformer 108 is encrypted, transmitted to a data server where the real-time DP measurements are periodically logged. The historical data which is stored in a non-volatile computer readable memory enables predictive maintenance, improves reliability analysis, and enhances diagnostics in the event of a failure.

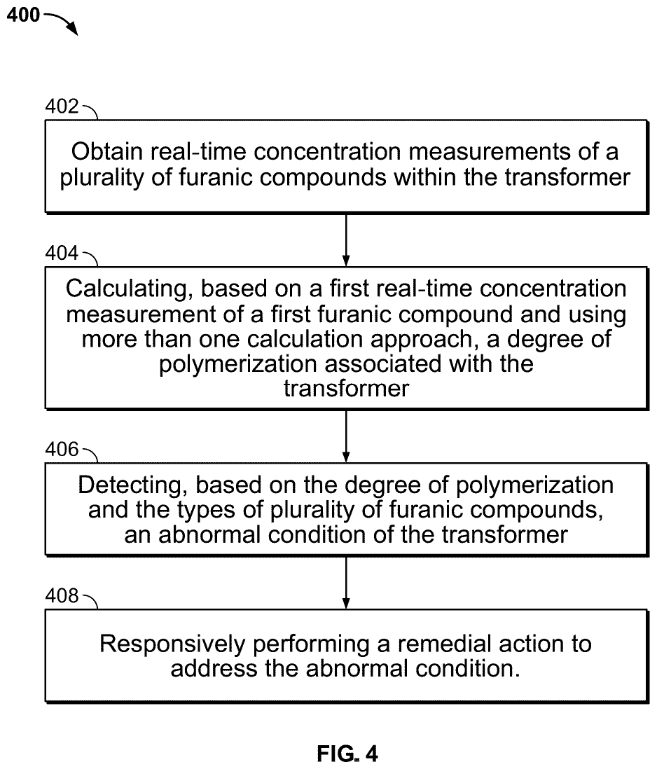

FIG. 4 is a flow diagram of an example method 400. For clarity of presentation, the description that follows generally describes process 400 in the context of the other figures in this description.

The system first obtains 402 real-time concentration measurements of a plurality of furanic compounds present within the transformer 108 oil. The furanic sensors 110 are configured to detect the presence of at least one of the following compounds: Furaldehyde (2FAL), 5-Methyl-2-Furaldehyde (5M2F), 2-Acetylfuran (2ACF), 5 Hydroxymethyl-2-Furaldehyde (5H2F), 2-Furfuryl Alcohol (2FOL). These furanic compounds correspond to relative parameters of a transformer's condition. Specifically, 2FAL corresponds to overheating and normal aging, 5M2F corresponds to high temperatures, 2ACF corresponds to rare and undefined causes, 5H2F corresponds to oxidation, and 2FOL corresponds to high moisture. The step may also include conducting a dissolved gas analysis within the transformer 108 oil to detect the presence of gases such as carbon dioxide (CO₂) and carbon monoxide (CO).

The system then calculates 404 the degree of polymerization of the transformer by using the measured concentration of furanic compounds as input in more than one calculation approach. These approaches may include a Chendong approach, a Stebbin approach, a first Myers approach, a second Myers approach, an Arrhenius approach, or a Pablo approach as described above.

The system then detects 406 the status and health of the transformer and detects the presence of abnormal conditions within the transformer 108 based on one or more calculation methods described in step 404. This step may also include transmitting the calculation result to the local display 102 and/or to a remote control room.

The system then performs 408 remedial actions to address any detected abnormal conditions. The remedial actions may be in the form of generating alerts for overdue maintenance or failures detected by any of the monitoring system components.

In some implementations, the real-time concentration measurements are obtained from at least one local furanic sensor installed within the transformer.

In some implementations, the method is performed by an embedded system including a microcontroller, a local display, and a communication device.

In some implementations, the plurality of furanic compounds comprise: 2 Furaldehyde (2FAL), 5-Methyl-2-Furaldehyde (5M2F), 2-Acetylfuran (2ACF), 5 Hydroxymethyl-2-Furaldehyde (5H2F), 2-Furfuryl Alcohol (2FOL).

In some implementations, the first furanic compound is 2 Furaldehyde (2FAL).

In some implementations, the more than one calculation approach includes more than one of: a Chendong approach, a Stebbin approach, a first Myers approach, or a second Myers approach.

In some implementations, the Chendong approach includes calculating the degree of polymerization as:

$$D P = \frac{1.51 - {log}_{10} ( {C}_{f u r} )}{0.0035} ,$$

where C_{fur} is the first concentration of 2FAL in parts per million (ppm).

In some implementations, the Stebbin approach includes calculating the degree of polymerization as:

$$D P = \frac{1.5655 - {log}_{10} ( {C}_{f u r} )}{0.0035} ,$$

where C_{fur} is the first concentration of 2FAL in parts per million (ppm).

In some implementations, the first Myers approach includes calculating the degree of polymerization as:

$$D P = - 343.8 * {log}_{10} ( {C}_{f u r} ) + 1387.5 ,$$

where C_{fur} is the first concentration of 2FAL in parts per billion (ppb).

In some implementations, the second Myers approach includes calculating the degree of polymerization as:

$$D P = - 285.7 * {log}_{10} ( {C}_{f u r} * 0.88 ) + 1288.6 ,$$

where C_{fur} is the first concentration of 2FAL in parts per billion (ppb).

In some implementations, the remedial action includes at least one of: removing the transformer from service, outputting an audible alert, outputting an alert on a display device, or adjusting operation of the transformer.

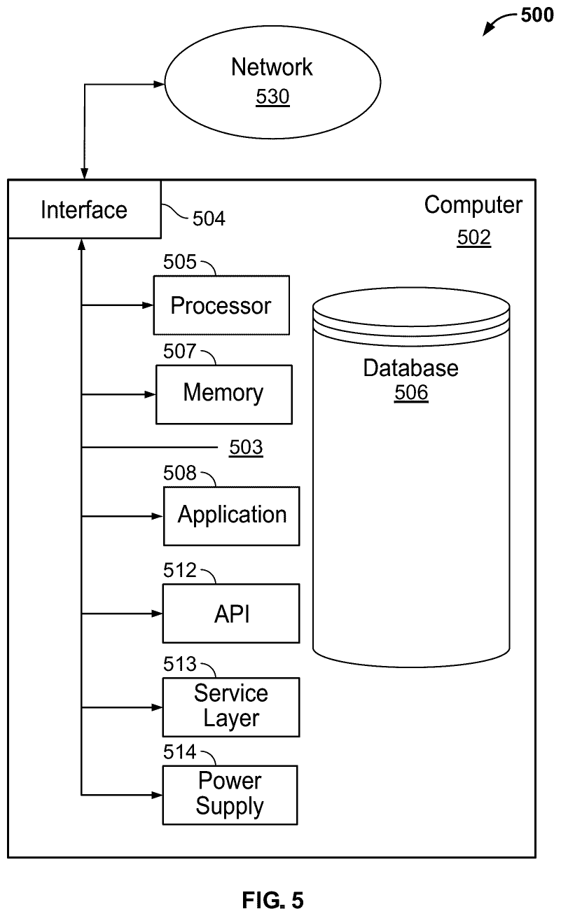

FIG. 5 is a block diagram of an example computer system 500 used to provide computational functionalities associated with described algorithms, methods, functions, processes, flows, and procedures described in the present disclosure, according to some implementations of the present disclosure. The illustrated computer 502 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 502 can include input devices such as keypads, keyboards, and touch screens that can accept user information. Also, the computer 502 can include output devices that can convey information associated with the operation of the computer 502. The information can include digital data, visual data, audio information, or a combination of information. The information can be presented in a graphical user interface (UI) (or GUI).

The computer 502 can serve in a role as a client, a network component, a server, a database, a persistency, or components of a computer system for performing the subject matter described in the present disclosure. The illustrated computer 502 is communicably coupled with a network 524. In some implementations, one or more components of the computer 502 can be configured to operate within different environments, including cloud-computing-based environments, local environments, global environments, and combinations of environments.

At a high level, the computer 502 is an electronic computing device operable to receive, transmit, process, store, and manage data and information associated with the described subject matter. According to some implementations, the computer 502 can also include, or be communicably coupled with, an application server, an email server, a web server, a caching server, a streaming data server, or a combination of servers.

The computer 502 can receive requests over network 524 from a client application (for example, executing on another computer 502). The computer 502 can respond to the received requests by processing the received requests using software applications. Requests can also be sent to the computer 502 from internal users (for example, from a command console), external (or third) parties, automated applications, entities, individuals, systems, and computers.

Each of the components of the computer 502 can communicate using a system bus 504. In some implementations, any or all of the components of the computer 502, including hardware or software components, can interface with each other or the interface 506 (or a combination of both), over the system bus 504. Interfaces can use an application programming interface (API) 514, a service layer 516, or a combination of the API 514 and service layer 516. The API 514 can include specifications for routines, data structures, and object classes. The API 514 can be either computer-language independent or dependent. The API 514 can refer to a complete interface, a single function, or a set of APIs.

The service layer 516 can provide software services to the computer 502 and other components (whether illustrated or not) that are communicably coupled to the computer 502. The functionality of the computer 502 can be accessible for all service consumers using this service layer. Software services, such as those provided by the service layer 516, can provide reusable, defined functionalities through a defined interface. For example, the interface can be software written in JAVA, C++, or a language providing data in extensible markup language (XML) format. While illustrated as an integrated component of the computer 502, in alternative implementations, the API 514 or the service layer 516 can be stand-alone components in relation to other components of the computer 502 and other components communicably coupled to the computer 502. Moreover, any or all parts of the API 514 or the service layer 516 can be implemented as child or sub-modules of another software module, enterprise application, or hardware module without departing from the scope of the present disclosure.

The computer 502 includes an interface 506. Although illustrated as a single interface 506 in FIG. 5, two or more interfaces 506 can be used according to implementations of the computer 502 and the described functionality. The interface 506 can be used by the computer 502 for communicating with other systems that are connected to the network 524 (whether illustrated or not) in a distributed environment. Generally, the interface 506 can include, or be implemented using, logic encoded in software or hardware (or a combination of software and hardware) operable to communicate with the network 524. More specifically, the interface 506 can include software supporting one or more communication protocols associated with communications. As such, the network 524 or the interface's hardware can be operable to communicate physical signals within and outside of the illustrated computer 502.

The computer 502 includes a processor 508. Although illustrated as a single processor 508 in FIG. 5, two or more processors 508 can be used according to implementations of the computer 502 and the described functionality. Generally, the processor 508 can execute instructions and can manipulate data to perform the operations of the computer 502, including operations using algorithms, methods, functions, processes, flows, and procedures as described in the present disclosure.

The computer 502 also includes a database 520 that can hold data (such geomechanics data 522) for the computer 502 and other components connected to the network 524 (whether illustrated or not). For example, database 520 can be in-memory or a database storing data consistent with the present disclosure. In some implementations, database 520 can be a combination of two or more different database types (for example, hybrid in-memory and conventional databases) according to implementations of the computer 502 and the described functionality. Although illustrated as a single database 520 in FIG. 5, two or more databases (of the same, different, or combination of types) can be used according to implementations of the computer 502 and the described functionality. While database 520 is illustrated as an internal component of the computer 502, in alternative implementations, database 520 can be external to the computer 502.

The computer 502 also includes a memory 510 that can hold data for the computer 502 or a combination of components connected to the network 524 (whether illustrated or not). Memory 510 can store any data consistent with the present disclosure. In some implementations, memory 510 can be a combination of two or more different types of memory (for example, a combination of semiconductor and magnetic storage) according to implementations of the computer 502 and the described functionality. Although illustrated as a single memory 510 in FIG. 5, two or more memories 510 (of the same, different, or combination of types) can be used according to implementations of the computer 502 and the described functionality. While memory 510 is illustrated as an internal component of the computer 502, in alternative implementations, memory 510 can be external to the computer 502.

The application 512 can be an algorithmic software engine providing functionality according to implementations of the computer 502 and the described functionality. For example, application 512 can serve as one or more components, modules, or applications. Further, although illustrated as a single application 512, the application 512 can be implemented as multiple applications 518 on the computer 502. In addition, although illustrated as internal to the computer 502, in alternative implementations, the application 512 can be external to the computer 502.

The computer 502 can also include a power supply 518. The power supply 518 can include a rechargeable or non-rechargeable battery that can be configured to be either user- or non-user-replaceable. In some implementations, the power supply 518 can include power-conversion and management circuits, including recharging, standby, and power management functionalities. In some implementations, the power-supply 518 can include a power plug to allow the computer 502 to be plugged into a wall socket or a power source to, for example, power the computer 502 or recharge a rechargeable battery.

There can be any number of computers 502 associated with, or external to, a computer system including the computer 502, with each computer 502 communicating over network 524. Further, the terms “client,” “user,” and other appropriate terminology can be used interchangeably, as appropriate, without departing from the scope of the present disclosure. Moreover, the present disclosure contemplates that many users can use one computer 502 and one user can use multiple computers 502.

Implementations of the subject matter and the functional operations described in this disclosure can be implemented in digital electronic circuitry, in tangibly embodied computer software or firmware, in computer hardware, including the structures disclosed in this disclosure and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatus, devices, and machines for processing data, including by way of example, a programmable processor, a computer, or multiple processors or computers. The apparatus can also include special purpose logic circuitry including, for example, a central processing unit (CPU), a field programmable gate array (FPGA), or an application specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, for example LINUX, UNIX, WINDOWS, MAC OS, ANDROID, or IOS.

The methods, processes, or logic flows described in this disclosure can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as RAM, read only memory (ROM), phase change memory (PRAM), static random-access memory (SRAM), dynamic random-access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks.

While this disclosure contains many specific implementation details, these should not be construed as limitations on the scope of what may be claimed, but rather as descriptions of features that may be specific to implementations. Certain features that are described in this disclosure in the context of separate implementations can also be implemented, in combination or in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Several implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium.

Several embodiments of these systems and methods have been described. Nevertheless, it will be understood that various modifications may be made without departing from the spirit and scope of this disclosure. Accordingly, other embodiments are within the scope of the following claims.

We claim:

1. A method for real-time monitoring of the health of a transformer comprising:
obtaining real-time concentration measurements of a plurality of furanic compounds within the transformer;
calculating, based on a first real-time concentration measurement of a first furanic compound and using more than one calculation approach, a degree of polymerization associated with the transformer;
detecting, based on the degree of polymerization and the types of plurality of furanic compounds, an abnormal condition of the transformer; and
responsively performing a remedial action to address the abnormal condition.

2. The method of claim 1, wherein the real-time concentration measurements are obtained from at least one local furanic sensor installed within the transformer.

3. The method of claim 1, wherein the method is performed by an embedded system comprising a microcontroller, a local display, and a communication device.

4. The method of claim 1, wherein the plurality of furanic compounds comprise: 2 Furaldehyde (2FAL), 5-Methyl-2-Furaldehyde (5M2F), 2-Acetylfuran (2ACF), 5 Hydroxymethyl-2-Furaldehyde (5H2F), 2-Furfuryl Alcohol (2FOL).

5. The method of claim 1, wherein the first furanic compound is 2 Furaldehyde (2FAL).

6. The method of claim 5, wherein the more than one calculation approach comprises more than one of: a Chendong approach, a Stebbin approach, a first Myers approach, or a second Myers approach.

7. The method of claim 6, wherein the Chendong approach comprises calculating the degree of polymerization as:
$$D P = \frac{1.51 - {log}_{10} ( {C}_{f u r} )}{0.0035} ,$$
where C_{fur} is the first concentration of 2FAL in parts per million (ppm).

8. The method of claim 6, wherein the Stebbin approach comprises calculating the degree of polymerization as:
$$D P = \frac{1.5655 - {log}_{10} ( {C}_{f u r} )}{0.0035} ,$$
where C_{fur} is the first concentration of 2FAL in parts per million (ppm).

9. The method of claim 6, wherein the first Myers approach comprises calculating the degree of polymerization as:
$$D P = - 343.8 * {log}_{10} ( {C}_{f u r} ) + 1387.5 ,$$
where C_{fur} is the first concentration of 2FAL in parts per billion (ppb).

10. The method of claim 6, wherein the second Myers approach comprises calculating the degree of polymerization as:
$$D P = - 285.7 * {log}_{10} ( {C}_{f u r} * 0.88 ) + 1288.6 ,$$
where C_{fur} is the first concentration of 2FAL in parts per billion (ppb).

11. The method of claim 1, wherein the remedial action comprises at least one of: removing the transformer from service, outputting an audible alert, outputting an alert on a display device, or adjusting operation of the transformer.

12. A system for real-time monitoring of the health of a transformer, the system comprising:
at least one processor;
a memory storing instructions that, when executed by the at least one processor, cause the at least one processor to perform operations comprising:
obtaining real-time concentration measurements of a plurality of furanic compounds within the transformer;
calculating, based on a first real-time concentration measurement of a first furanic compound and using more than one calculation approach, a degree of polymerization associated with the transformer;
detecting, based on the degree of polymerization and the types of plurality of furanic compounds, an abnormal condition of the transformer; and
responsively performing a remedial action to address the abnormal condition.

13. The system of claim 12, wherein the real-time concentration measurements are obtained from at least one local furanic sensor installed within the transformer.

14. The system of claim 12, further comprising:
an embedded system comprising a microcontroller, a local display, and a communication device.

15. The system of claim 12, wherein the plurality of furanic compounds comprise: 2 Furaldehyde (2FAL), 5-Methyl-2-Furaldehyde (5M2F), 2-Acetylfuran (2ACF), 5 Hydroxymethyl-2-Furaldehyde (5H2F), 2-Furfuryl Alcohol (2FOL).

16. The system of claim 12, wherein the first furanic compound is 2 Furaldehyde (2FAL).

17. The system of claim 12, wherein the more than one calculation approach comprises more than one of: a Chendong approach, a Stebbin approach, a first Myers approach, or a second Myers approach.

18. The system of claim 17, wherein the Chendong approach comprises calculating the degree of polymerization as:
$$D P = \frac{1.51 - {log}_{10} ( {C}_{f u r} )}{0.0035} ,$$
where C_{fur} is the first concentration of 2FAL in parts per million (ppm).

19. The system of claim 17, wherein the Stebbin approach comprises calculating the degree of polymerization as:
$$D P = \frac{1.5655 - {log}_{10} ( {C}_{f u r} )}{0.0035} ,$$
where C_{fur} is the first concentration of 2FAL in parts per million (ppm).

20. A non-transitory computer storage medium encoded with instructions that, when executed by one or more computers, cause the one or more computers to perform operations for real-time monitoring of the health of a transformer, the operations comprising:
obtaining real-time concentration measurements of a plurality of furanic compounds within the transformer;
calculating, based on a first real-time concentration measurement of a first furanic compound and using more than one calculation approach, a degree of polymerization associated with the transformer;
detecting, based on the degree of polymerization and the types of plurality of furanic compounds, an abnormal condition of the transformer; and
responsively performing a remedial action to address the abnormal condition.
